(19) United States
(12) Patent Application Publication
(10) Pub. No.: US 2024/0029817 A1
(43) Pub. Date: Jan. 25, 2024

(54) Digital Model of the Human

(71) Applicant: Anders Kaare Norgaard, Copenhagen (DK)

(72) Inventor: Anders Kaare Norgaard, Copenhagen (DK)

(21) Appl. No.: 18/374,292

(22) Filed: Sep. 28, 2023

(51) Int. Cl. G16B 5/00

(52) U.S. Cl. CPC G16B 5/00

(57) ABSTRACT

The present disclosure provides an approximation method for the functionality managed by genes of the human body, so that it estimates the human functions and consequences, when one or more factors are changed. The invention aggregates the areas of metabolization and signaling in one consistent and coherent approximation. The genes, which rule these areas constitute only approx. 1% of the DNA, so there is evidently more human functionality to be discovered. But the invention ties the known areas consistently together. And it joins the existence of a feedback mechanism to the metabolization and signaling areas to enable predictions of outcomes as a result of changes in inputs. The invention provides new insights, because it manages the cross-human effects, including whole-body causalities far apart in separate pathways and when a gene affects more than one of the areas mentioned above.

### BACKGROUND/SUMMARY

### Background of the Invention

### Background Explanation

The two main functional areas of Metabolization and Signaling are described in FIG. 6. They are based on
Metabolization: Reactions (that transform substances into other substances)
Signaling: Receptors (that based on ligands (a subset of substances) trigger a cascade of events ending with the expression (transcription) of one or more Genes and in executing other functions on the way

The Metabolization Reactions are concatenated in Metabolization Pathways (where an output Substance of one Reaction is the input Substance of another Reaction)—each Reaction facilitated by one or more Enzymes/Genes. This is depicted in FIG. 7 where Pathways are themselves concatenated.

Not all Reactions are shown in FIG. 7. We estimate that there are in total approx. 2,200 Reactions.

An example of Signaling events is shown in FIG. 8. Normally a Signaling Pathway is a grouping of Signaling functions with a related outcome and which interact. They begin by a Receptor or several Receptors being activated by Ligands—and they end up in Expression (Transcription) of Genes.

The Receptors, of which we estimate a total of 800, are on the highest level of their hierarchy grouped into 5 types as seen on FIG. 9. In this figure is shown how the Receptors of 2 of these types relate to 2 other classifications (Membrane Transports and Transcription Factors).

The whole system of Metabolization and Signaling is related with Genes, Body Parts, DNA Damage and Mutation (and Repair), and the Immune System, and with visualizations of the two types of pathways.

### Prior Art

There is no consistent data model and no complete data set of all elements in the Human Model. In the following a detailed status is given. The databases or systems mentioned are listed below.

The Genes and the Substances are well recorded in databases (e.g. Genes in UniProt, Substances in PubChem).

Some mutations (instances of Genes, socalled Alleles) and how they act together in pairs (socalled Diplotypes) are recorded as well.

### (A): Metabolization

Human Metabolization is recorded in databases (in e.g. HumanCyc). It shows in basic process steps (called Reactions) how one or several Substances is/are converted to one or several other Substances catalyzed by one or several Genes (that act through Enzymes in a one-to-one relationship between Genes and Enzymes).

These Substances are either basic substances identified with a unique code in e.g. PubChem, or are a higher order substance (a group of basic substances) in a substance hierarchy defined in each data source.

The basic processes or Reactions are concatenated or otherwise combined into Metabolization Pathways (where each step of the pathway is a basic process).

Some sources (e.g. HumanCyc) do not include the Metabolization of drugs (exogenous substances), only of naturally occurring (endogeneous) substances.

The diagrams that appear in these systems (e.g. HumanCyc) correspond to data in and are generated from the underlying database, so the depicting functionality is totally data driven. Thereby all the elements and their relations are present in the data as well.

The data is complete in that all known metabolization reactions are there (in e.g. HumanCyc). Sometimes data may have to be added or refined.

The hierarchy of substances is at times not a strict hierarchy in that sometimes substances defined as elements in the hierarchy need parametrization (e.g. number of chain elements) to be fully specified—in which case you see the same substance being both input and output of a process, but with the removal or addition of one element of the substance. So to be precise and unique we need to add that parametrization to the substance in the hierarchy to define it.

### (B): Signaling

Signaling Pathways are a cascading combination that typically starts with a Ligand activating a Receptor, which then invokes a cascaded coupling of elements that end with a combination of two:
1. The Expression (through Transcription) of Genes, and
2. One or a set of functions typically described in words, that function sometimes covering so far unspecified signaling

Gene Expression covers the production of enzymes and other proteins mediated by said Genes.

Signaling elements and pathways are not depicted in HumanCyc. And in other systems, where they are, e.g. KEGG, there is no complete set of data in that many diagrams are drawn and appears as a drawing or diagram, meaning that it takes a human to interpret them, and there is much information given in the diagrams, that is not in data.

The signaling information is therefore
not all of it in data (in that the diagrams must be viewed and interpreted by a human—e.g. the arrows in the diagrams are drawn and appear in the diagram only, and the relation between the two elements that the arrow represents is not in data),
inconsistent with and not locked to the data model in a Metabolization source like HumanCyc. Some Signaling Pathways take as a starting point a substance produced in the middle of a Metabolization Pathways, without saying how the signaling interacts or how it branches off from the metabolization processes. E.g. the substance Serotonin is in the middle of a Metabolization Pathway, but it is also a Ligand that may start off a Signaling Pathway and therefore not be available to the last part of the Metabolization Pathway.

Signaling diagrams are furthermore incomplete in that not all signaling for all receptors is depicted in the diagrams. It is estimated that a source of Signaling Pathways like KEGG covers roughly 200 receptors out of the more than 800 receptors.

There are many diverse sources of information for signaling, with conflicting and inconsistent information. The hierarchies of the elements that partake in signaling are often separately (and sometimes conflictingly) recorded and in other systems than where the diagrams are. E.g. the hierarchy is specified by Wikipedia or in scientific papers, and the diagrams are specified in KEGG.

(A)+(B): Common issues with Metabolization and Signaling

In Metabolization as well as in Signaling databases there is no provision of any of the following elements
Timings or speed functions, giving how long time a reaction or process takes
Distribution functions when a graph branches out (e.g. when the substance Tryptophan can metabolize in two separate directions, what is then the distribution between the two branches)
Merging rules when a graph joins from several branches to one: Does the process await all branches, or just one, or a combination

### (C) Feedback Mechanisms.

There is not much data on “feedback regulation”, i.e. the downregulation of genes, when the resulting substances cf. Metabolization is in ample supply in the body.

There is evidence that this aspect is important to maintaining the stability of the body, but it is not explained and detailed down to what are the probable mechanisms behind it; the Signaling where the substance as a ligand exerts a downregulating function on the genes involved in its production.

The main effort of research is to find “forward” mechanisms, i.e. signaling pathways that have the feedback role of down-regulating genes, but the work has not come very far in representing the feedback mechanisms.

Furthermore this area may not be fully explained by the functional elements known to date (given that the genes only constitute approx. 1% of the total DNA, and we seem to look for gene regulated functionality only). It may be that some of the functionality that constitutes the “feedback regulation” is implemented in the parts that are currently not explained by science.

(A)+(B)+(C): Common Issues. The general perception of the problem described in this disclosure is governed by a structure that doesn't support a mechanistical data model of the area (i.e. usable in that it can be used to predict output and a new state as a result of changed inputs).

Quite often the area is specified using the following hierarchy of concepts (cf. FIG. 4):
Genomics
Transcriptomics
Proteomics
Metabolomics

The general view is that this is a biological issue primarily, with a lot of functionality that is hard to classify and “snap to concept”, i.e. devise approximations to reality and then adhere to these approximations in order to use the power of IT to predict outcomes.

There is a notion of “interactions” (between gene-governed proteins, i.e. the 20,000 existing in the human body, totaling 20,000 to the power of 2 or 400 million potential interactions)—and they are derived in a partially unscientific way e.g. by letting code (“AI”) browse through abstracts af scientific papers to see if a set of two gene abbreviations representing two proteins is mentioned in the same abstract, thereby concluding that they must interact—so the cause or causality of this interaction is unexplained, just given by a total “strength score”. This has given 13 million (out of the above mentioned 400 million possible) interactions recorded in the database String.

This is indeed a valid entry point when investigating a potential interaction further, but it is necessary to dive deeper to find out why there is a particular interaction recorded.

### List of Primary Databases:

| | Database | URL | Description |
|---|---|---|---|
| 1 | HumanCyc | HumanCyc.org | Developed by SRI International (a branch-off |
| | | Special case | from Stanford University, CA, USA) |
| | | (subset) for the | The database holds metabolization pathways |
| | | human body-more | in several organisms, hereunder the human. |
| | | comprehensive | HumanCyc is the subset relating to humans. |
| | | references: | More than 1 million processes (chemical |
| | | BioCyc.org | reactions) (16,031 biochemical reactions in |
| | | MetaCyc.org | MetaCyc), with reference to Substances being |
| | | | input and output respectively, and Enzymes |
| | | | (and therefore genes) catalyzing the process. |
| 2 | KEGG | www.kegg.jp/ | Kyoto Encyclopedia of Genes and Genomes |
| | | | (KEGG) is an extensive and widely used |
| | | | database. It is a manually curated source |
| | | | incorporating 18 databases classified into |
| | | | genomic, systems, health, and chemical data. |
| 3 | HMDB | hmdb.ca | The HMDB is a broad source delivering |
| | | | information about homo-sapiens metabolites |
| | | | and their associated physiological, chemical, |
| | | | and biological properties. To date, HMDB has |
| | | | 220,945 total metabolites. |
| | | | Linked to from SMPDB. Freely available. |
| | | | Links back to SMPDB when showing a |
| | | | pathway. |
| | | | HMDB contains over 41,000 metabolite entries |
| | | | including both water-soluble and lipid soluble |
| | | | metabolites as well as metabolites that would |
| | | | be regarded as either abundant (>1 uM) or |
| | | | relatively rare (<1 nM). Additionally, |
| | | | approximately 7,200 protein (and DNA) |
| | | | sequences are linked to these metabolite |
| | | | entries. |
| 4 | SMPDB | smpdb.ca/ | Small Molecule Pathway Database. |
| | | | Containing more than 30,000 small molecule |
| | | | pathways found in humans only. |
| | | | Driven by the University of Alberta, Edmonton, |
| | | | Alberta, Canada. |
| | | | SMPDB is a comprehensive, interactive, visual |
| | | | database that includes over 48,000 discovered |
| | | | pathways. Most of the pathways do not exist in |
| | | | other pathway databases. SMPDB helps in |
| | | | pathway discovery and interpretation in |
| | | | metabolomics, proteomics, transcriptomics, |
| | | | and systems biology. |
| 5 | Reactome | reactome.org/ | Founded in 2003, the Reactome project is led |
| | | | by Lincoln Stein of OICR [Ontario], Peter |
| | | | D'Eustachio of NYULMC [New York], Henning |
| | | | Hermjakob of EMBL-EBI [UK], and Guanming |
| | | | Wu of OHSU [Oregon]. |
| | | | The Reactome Knowledgebase is a distinct |
| | | | curated database of pathways and reactions in |
| | | | human biology, cross-referenced with several |
| | | | resources, such as essential literature and |
| | | | different pathway-related databases. It aims its |
| | | | manual annotation effort on Homo-sapiens, a |
| | | | single species, and applies a separate |
| | | | consistent data model within the whole biology |
| | | | domain. The Reactome describes a reaction |
| | | | as an event in biology that alters the condition |
| | | | of a biological molecule. Degradation, |
| | | | activation, binding, translocation, and typical |
| | | | biochemical events, including a catalyst, are |
| | | | reactions. It presents molecular features of |
| | | | signal transduction, transport, metabolism, |
| | | | DNA replication, and more cellular activities. It |
| | | | contains 2546 human pathways and 1940 |
| | | | small molecules |
| 6 | PubChem | pubchem.ncbi.nlm. | Definition of all chemical substances (the |
| | | nih.gov/ | bottom elements of all the substance |
| | | | hierarchies or ontologies). Holds appr. 60 |
| | | | million substances. |
| | | | Used to uniquely identify all substances by |
| | | | their PubChem ID, when they are real (as |
| | | | opposed to up in the hierarchy). |
| 7 | UniProt | www.uniprot.org/ | Database of all genes (and their enzymes). |
| | | | Used to uniquely define all genes (via their |
| | | | name and UniProt ID). |
| | | | It has interactions recorded between genes, |
| | | | without explaining the nature of these |
| | | | interactions. E.g. between the genes AR |
| | | | (androgen receptor) and DDC: The interaction |
| | | | being from other sources, that DDC is a |
| | | | coactivator of AR. |
| 8 | DrugBank | go.drugbank.com/ | Used from time to time, the primary link is |
| | | | Wikipedia. Explaining the details of a drug. |
| | | | Contains over 7,800 drug entries, nearly 2,200 |
| | | | FDA-approved small molecule drugs, 340 |
| | | | FDA-approved biotech (protein/peptide) drugs, |
| | | | 93 nutraceuticals and >5,000 experimental |
| | | | drugs. Additionally, more than 3,500 non- |
| | | | redundant protein (i.e. drug target) sequences |
| | | | are linked to these FDA approved drug entries. |
| | | | Each DrugCard entry contains more than 100 |
| | | | data fields with half of the information being |
| | | | devoted to drug/chemical data and the other |
| | | | half devoted to drug target or protein data. |
| 9 | Depression | menda.cqmu.edu.cn: | Metabolite Network of Depression Database |
| | | 8080/index.php | (MENDA) is a broad metabolite-disease |
| | | | association database that integrates all |
| | | | existing knowledge and datasets of metabolic |
| | | | characterization in depression. In addition, |
| | | | study and tissue type, organism, category of |
| | | | depression, sample size, platform (MS-based, |
| | | | MRS, NMR), and differential metabolites are |
| | | | provided. |
| 10 | BiGG | bigg.ucsd.edu/ | BiGG Models is a biochemical, genetic, and |
| | | | genomic knowledge base of genome-scale |
| | | | metabolic network reconstructions. BiGG |
| | | | Models includes more than 75 superior, |
| | | | manually curated genome-scale metabolic |
| | | | models. It also delivers a broad application |
| | | | interface for accessing BiGG Models with |
| | | | modeling and analysis kits. In addition, |
| | | | reaction and metabolite identifiers and pathway |
| | | | visualization were formalized in BiGG Models. |
| 11 | BRENDA | www.brenda- | The Braunschweig Enzyme Database |
| | | enzymes.org/ | (BRENDA) enzyme database contains |
| | | | comprehensive functional enzyme and |
| | | | metabolism data such as measured kinetic |
| | | | parameters. The main part has more than 5 |
| | | | million data points for almost 90,000 enzymes. |
| | | | In addition, BRENDA presents accessible |
| | | | enzyme information from fast to superior text- |
| | | | and structured-based searches for word maps, |
| | | | enzyme-ligand interactions, and enzyme data |
| | | | visualization. |
| 12 | ChEBI | www.ebi.ac.uk/chebi | ChEBI is an open-access glossary of molecular |
| | | | entities aimed at small biochemical |
| | | | compounds. |
| 13 | Chem | chemspider.com/ | ChemSpider is a freely accessible chemical |
| | Spider | | structure database delivering a quick structure |
| | | | and text search covering over one hundred |
| | | | million structures from hundreds of data |
| | | | resources. |
| 14 | Metabo | www.ebi.ac.uk/ | MetaboLights is a database that includes |
| | Lights | metabolights | metabolomics studies research, raw |
| | | | experimental data, and related metadata. |
| | | | MetaboLights is cross-technique and cross- |
| | | | species and includes metabolite structures and |
| | | | their related biological roles, reference spectra, |
| | | | concentrations and locations, and metabolic |
| | | | experiments data. Users can upload their |
| | | | research datasets into the MetaboLights |
| | | | Repository. Researchers are then |
| | | | automatically given a unique and stable |
| | | | identifier for publication reference. |
| 15 | Metabolomics | metabolomicsworkbench. | The Metabolomics Workbench is a public |
| | Work | org/ | repository for experimental metabolomics |
| | bench | | metadata and data covering several species |
| | | | and experimental platforms, metabolite |
| | | | structures, metabolite standards, tutorials, |
| | | | protocols, training material, and more |
| | | | educational resources. It can combine, |
| | | | examine, deposit, track, and distribute big |
| | | | heterogeneous data from many MS-and NMR- |
| | | | based metabolomics studies. It covers over |
| | | | twenty diverse species, including humans and |
| | | | other mammals, insects, invertebrates, plants, |
| | | | and microorganisms. |
| 16 | MetSigDis | www.bio- | MetSigDis is a free web-based tool that offers |
| | | annotation.cn/ | a comprehensive metabolite alterations |
| | | MetSigDis/ | resource in various diseases. The database |
| | | | deposited 6849 curated associations between |
| | | | 2420 metabolites and 129 diseases among |
| | | | eight species, including humans and model |
| | | | organisms. |
| 17 | Virtual | www.vmh.life/ | Virtual Metabolic Human is a web-based |
| | Metabolic | | database capturing the knowledge of Homo- |
| | Human | | sapiens metabolism within 5 interlinked |
| | | | resources, including, Homo-sapiens |
| | | | metabolism, Disease, Gut microbiome, |
| | | | ReconMaps, and Nutrition. The VMH's |
| | | | exceptional features are (i) the introduction of |
| | | | the metabolic reconstructions of Homo-sapiens |
| | | | and gut microbes for metabolic modeling; (ii) |
| | | | seven Homo-sapiens metabolic maps for data |
| | | | visualization; (iii) a nutrition designer; (iv) an |
| | | | accessible web page and application user |
| | | | interface to access the content; (v) feedback |
| | | | option for community users' interactions and |
| | | | (vi) the linking of its entities to 57 web |
| | | | resources. |
| 18 | Wiki | wikipathways.org/ | WikiPathways is a reliable and rich pathway |
| | Pathways | | database that captures biological pathways' |
| | | | collective knowledge. By delivering a database |
| | | | in a curated, machine-readable system, |
| | | | visualization and omics data studies is |
| | | | supported. |
| 19 | RaMP | github.com/mathelab/ | The relational database of Metabolomics |
| | | RaMP-DB/ | Pathways (RaMP) is a public database to |
| | | | combine biological pathways from the |
| | | | WikiPathways, KEGG Reactome, and the |
| | | | HMDB. RaMP maps metabolites and genes to |
| | | | biochemical and disease pathways and can be |
| | | | incorporated into other existing software. It can |
| | | | be used as a stand-alone resource |
| | | | (https://github.com/mathelab/RaMP-DB/, |
| | | | accessed on 1 Apr. 2022) or incorporated into |
| | | | other tools (https://github.com/mathelab/RaMP- |
| | | | DB/inst/extdata/, accessed on 1 Apr. 2022). |
| 20 | Pathway | www.pathwaycommons. | Pathway Commons is one of the most |
| | Commons | org/ | extensive composite databases. It is an |
| | | | integrated resource of openly accessible |
| | | | information about biological pathways involving |
| | | | biochemical reactions, transport and catalysis |
| | | | events, assembly of biomolecular complexes, |
| | | | and physical interactions, including DNA, RNA, |
| | | | proteins, and small molecules such as drug |
| | | | compounds and metabolites. |
| 21 | BMRB | www.bmrb.wisc.edu | A variety of databases stands as a |
| | | | metabolomics dataset repository. To mention |
| | | | some, BioMagResBank (BMRB) is a public |
| | | | repository for NMR spectroscopy data from |
| | | | peptides, proteins, nucleic acids, and more |
| | | | biomolecules. In addition, the Golm |
| | | | Metabolome Database (GMD) |
| | | | (http://gmd.mpimp-golm.mpg.de/) provides |
| | | | datasets for biologically quantified active |
| | | | metabolites and text search capabilities for |
| | | | GC-MS data. Moreover, the Mass Spectral |
| | | | Library (https://www.NIST.gov/srd/NIST- |
| | | | standard-referencedatabase-1a) extensively |
| | | | collects EI MS, MS/MS, replicate spectra, and |
| | | | retention index datasets. Finally, the Spectral |
| | | | Database System (SDBS) |
| | | | (https://sdbs.db.aist.go.jp/, accessed on 1 Apr. |
| | | | 2022) is a spectral database for organic |
| | | | compounds and has various MS, NMR, IR, |
| | | | Raman, ESR datasets. |
| 22 | Signor | signor.uniroma2.it | The SIGnaling Network Open Resource |
| | | | Entity types: |
| | | | Protein-7419, Chemical-1004, etc |
| | | | Mechanisms: |
| | | | Phosphorylation-10687, Binding-8699, |
| | | | Transcriptional regulation-3756, etc. |
| | | | Total: 35,000+ interactions |
| 23 | String | String-db.org | Consortium: Swiss Institute of Bioinformatics- |
| | | | Uni Zurich-Novo Nordisk Foundation Center |
| | | | Protein Research-European Molecular |
| | | | Biology Laboratory (Heidelberg) |
| 24 | BioGrid | TheBioGrid.org | The Biological General Repository for |
| | | | Interaction Datasets (BioGRID) is a public |
| | | | database that archives and disseminates |
| | | | genetic and protein interaction data from model |
| | | | organisms and humans (thebiogrid.org). |
| | | | BioGRID currently holds over 1,740,000 |
| | | | interactions curated from both high-throughput |
| | | | datasets and individual focused studies, as |
| | | | derived from over 70,000+ publications in the |
| | | | primary literature. |
| | | | Mainly people from Toronto, CA. |
| 25 | Pharm Var | pharmvar.org | More extensive information on each allele. |
| | | | The major focus of PharmVar is to catalogue |
| | | | allelic variation of genes impacting drug |
| | | | metabolism. |
| 26 | Pharm | pharmgkb.org | Combinations of alleles into diplotypes (pairs of |
| | GKB | | alleles as they appear in humans) and the |
| | | | corresponding metabolization |
| | | | Also pathways and metabolization database |

Other databases include:

AmiGO, BIND, BioCarta, BioGPS, CAZy, CDD, COG, COMPARTMENTS, CTD, DAVID, DGIdb, DisGeNet, eDGAR, EndoNet, Ensembl, Entrez, ExPASy, Expression Atlas, GAD, Gene Expression Omnibus, Gene Ontology, GeneWiki, GoGene, GXD, HAPMAP, HMGD, HOGENOM, HSLS, HUGO, ImmunoDB, iPathwayGuide, KOG, the Human Protein Atlas, LHDGN, LocDB, LOCATE, MalaCards, METAGENE, MGD, MGI, MouseMine, NCBI, NetDecoder, OMIM, OMMBID, OrthoDB, PANTHER, PathJam, Pathguide, Pathway Commons, Pfam, photon, Phyre2, PSORTdb, PID, PRK, ProDom, PROFESS, PROSITE, RefSeq, SIFT, SMART, SPATIAL, SuperTarget, Swiss-MODEL, Swiss-Prot, TIGR, Treefam, and TTD.

### U.S. Patent Documents

| Pat no | Date | Title | Assignee | |
|---|---|---|---|---|
| 7308363 | 2007 Dec. 11 | Modeling And | SRI International | reactant-product |
| | | Evaluation Metabolic | [Peter Karp] | relationships |
| | | Reaction Pathways And | | |
| | | Culturing Cells | | |
| 11673959 | 2023 Jun. 13 | Coiled Coil | THE SCRIPPS | Vaccine, |
| | | Immunoglobulin Fusion | RESEARCH | palivizumab |
| | | Proteins And | INSTITUTE, La | |
| | | Compositions Thereof | Jolla, CA | |
| 7724267 | 2010 May 25 | Systems, Methods And | Symyx Solutions, | Chemical |
| | | Computer Program | Inc, Sunnyvale, | synthesis |
| | | Products For | CA | |
| | | Determining Parameters | | |
| | | For Chemical Synthesis | | |

### U.S. Patent Applications

| Doc no | Date | Title | Assignee | About |
|---|---|---|---|---|
| US | 2018 Nov. 29 | METHOD AND SYSTEM | [SF and | |
| 20180342322 | | FOR | Chile] | |
| A1 | | CHARACTERIZATION | | |
| | | FOR APPENDIX- | | |
| | | RELATED CONDITIONS | | |
| | | ASSOCIATED WITH | | |
| | | MICROORGANISMS | | |
| US | 2019 Mar. 14 | METHOD AND SYSTEM | [SF and | |
| 20190078142 | | FOR | Chile] | |
| A1 | | CHARACTERIZATION | | |
| | | FOR FEMALE | | |
| | | REPRODUCTIVE | | |
| | | SYSTEM-RELATED | | |
| | | CONDITIONS | | |
| | | ASSOCIATED WITH | | |
| | | MICROORGANISMS | | |
| US | 2018 Sep. 27 | MODULAR ORGAN | [Boston, | cell culture |
| 20180272346 | | MICROPHYSIOLOGICAL | MA] | systems |
| A1 | | SYSTEM WITH | | |
| | | MICROBIOME | | |
| US | 2017 Oct. 26 | METHOD AND SYSTEM | [SF] | sequencing, |
| 20170308669 | | FOR MICROBIAL | | antibiotics |
| A1 | | PHARMACOGENOMICS | | |
| US | 2022 Jul. 21 | Metabolite Delivery For | [Tempe, | Drug delivery |
| 20220226499 | | Modulating Metabolic | AZ] | carriers. |
| A1 | | Pathways Of Cells | | Specific for |
| | | | | immune |
| | | | | diseases |
| US | 2022 Nov. 3 | METHODS FOR | [China] | Liquid |
| 20220349891 | | IDENTIFYING CANCER | | biopsies |
| A1 | | | | |
| US | 2022 Dec. 8 | ENGINEERED | [China] | Immune |
| 20220389398 | | CRISPR/CAS13 SYSTEM | | system |
| A1 | | AND USES THEREOF | | |
| US | 2023 Jun. 8 | COMPOSITIONS AND | [MO] | Aging, |
| 20230172232 | | METHODS USING AN | | mitochondrion |
| A1 | | AMINO ACID BLEND | | |
| | | FOR PROVIDING A | | |
| | | HEALTH BENEFIT IN AN | | |
| | | ANIMAL | | |
| US | 2020 Nov. 26 | IN-VITRO MODEL OF | [Germany] | Diagnosing |
| 20200370005 | | THE HUMAN GUT | | metabolic |
| A1 | | MICROBIOME AND | | diseases. |
| | | USES THEREOF IN THE | | Predict drug |
| | | ANALYSIS OF THE | | action. |
| | | IMPACT OF | | Bacterial panel, |
| | | XENOBIOTICS | | enzymatic |
| | | | | coverage |
| US | 2020 Jun. 25 | NASAL-RELATED | [SF] | nasal-related |
| 20200202979 | | CHARACTERIZATION | | characterization |
| A1 | | ASSOCIATED WITH THE | | |
| | | NOSE MICROBIOME | | |
| US | 2022 Dec. 22 | INTRAVENOUS | [IL] | IV pumps |
| 20220401640 | | INFUSION PUMPS WITH | | |
| A1 | | SYSTEM AND | | |
| | | PHARMACODYNAMIC | | |
| | | MODEL ADJUSTMENT | | |
| | | FOR DISPLAY AND | | |
| | | OPERATION | | |
| US | 2008 Dec. 25 | Compositions And | [FL] | Statin side |
| 20080318218 | | Methods For Inferring An | | effect: muscles |
| A1 | | Adverse Effect In | | |
| | | Response To A Drug | | |
| | | Treatment | | |
| US | 2017 Jun. 15 | HUMAN HEPATIC 3D | [Belgium] | 3D model, liver |
| 20170166870 | | CO-CULTURE MODEL | | |
| A1 | | AND USES THEREOF | | |

### Foreign Patent Documents

### [None].

### Other References

| | URL | Title | Author(s) |
|---|---|---|---|
| 1 | https://www.ncbi. | Survey for Computer-Aided Tools and | Bayan Hassan |
| | nlm.nih.gov/pmc/ | Databases in Metabolomics | Banimfreg, Abdulrahim |
| | articles/ | | Shamayleh, and Hussam |
| | PMC9610953/ | | Alshraideh |
| 2 | https://encyclopedia. | Databases in Metabolomics | |
| | pub/entry/31304 | | |
| 3 | https://elifesciences. | DNA damage-how and why we | Matt Yousefzadeh, |
| | org/articles/ | age? | Chathurika Henpita, |
| | 62852#:~:text=DNA | [Jan. 29, 2021] | Rajesh Vyas, Carolina |
| | %20damage%20 | | Soto-Palma, Paul |
| | contributes%20to | | Robbins, Laura |
| | %20aging,undamaged | | Niedernhofer |
| | %20cells%20 | | [Institute on the Biology |
| | through%20 | | of Aging and Metabolism |
| | their%20SASP | | Department of |
| | | | Biochemistry, Molecular |
| | | | Biology and Biophysics, |
| | | | University of Minnesota, |
| | | | United States] |
| 4 | https://bio.libretexts. | Control of Metabolism Through | [2.7.1 in a book] |
| | org/Bookshelves/ | Enzyme Regulation | |
| | Microbiology/ | [One of the main diagrams is from [5] | |
| | Microbiology_ | below] | |
| | (Boundless)/02%3A_ | | |
| | Chemistry/2.07 | | |
| | %3A_Enzymes/ | | |
| | 2.7.01%3A_Control_ | | |
| | of_Metabolism | | |
| | Through_Enzyme_ | | |
| | Regulation | | |
| 5 | https://openoregon. | Feedback Inhibition in Metabolic | [Section in “Principles of |
| | pressbooks. | Pathways | Biology”] |
| | pub/mhccmajorsbio/ | | |
| | chapter/6-7- | | |
| | feedback- | | |
| | inhibition-in- | | |
| | metabolic- | | |
| | pathways/ | | |
| 6 | https://www.ncbi. | Melatonin-A New Prospect in | Comfort Anim-Koranteng |
| | nlm.nih.gov/pmc/ | Prostate and Breast Cancer | Hira E Shah, 1 Nitin |
| | articles/ | Management | Bhawnani, Aarthi |
| | PMC8525668/ | [2021] | Ethirajulu, Almothana |
| | | | Alkasabera, Chike B |
| | | | Onyali, and Jihan A |
| | | | Mostafa |
| | | | [California Institute of |
| | | | Behavioral |
| | | | Neurosciences & |
| | | | Psychology, USA] |
| 7 | https://www.nature. | Serotonin regulates prostate growth | Emanuel Carvalho-Dias, |
| | com/articles/ | through androgen receptor | Alice Miranda, Olga |
| | s41598-017-15832- | modulation | Martinho, Paulo Mota, |
| | 5 | [2017] | Angela Costa, Cristina |
| | | | Nogueira-Silva, Rute S. |
| | | | Moura, Natalia Alenina, |
| | | | Michael Bader, Riccardo |
| | | | Autorino, Estêvão Lima & |
| | | | Jorge Correia-Pinto |
| | | | [University of Minho, |
| | | | Portugal] |

### BRIEF SUMMARY OF THE INVENTION

The Invention
approximates the metabolization and signaling functions of the human body, which are governed by the genes, and adds
a feedback mechanism that represents so far unspecified functionality including signaling, which ensures stability of the human body.

It does so by approximating in one consistent representation the above-mentioned areas, so that it is possible to make cross-human predictions based on changed inputs by using causalities and parameters.

This approximation introduces a model that spans Genes, Metabolization Reactions, Substances and their hierarchies, how the Genes partake in the Reactions and what Substances are inputs and outputs respectfully of a Reaction, how the Substances proceed with other Metabolizations Reactions or act as Ligands to Receptors and thereby trigger Signaling Pathways—or how Genes can play this role, when recognized Ligands are Proteins that are controlled directly by the Genes, not indirectly through Metabolization—and then how the Signaling Pathways, ending with transcription of other Genes than those that control the particular process, can be represented in data.

The invention furthermore includes statistical models of DNA replication errors, DNA repair, and functions to kill cells that bypass DNA repair with a “bad” mutation, and it takes into account known mutations that can be inherited and their known effects on metabolization and signaling and on DNA repair (e.g. the BRCA mutations that affect DNA repair). The invention takes into account that mutations act through Alleles (instances of Genes) and Diplotypes (pairs of Alleles)—and assume that it is the Diplotype that manages how a Gene governs its Enzymes and Proteins.

Thereby the approximation holds a way to represent the full cycle from Genes and their mutations back to Genes, and thereby a major part of the human body functions. It is supplemented by word descriptions in cases where we don't yet know the detailed functioning of signaling. This we refer to as (A) and (B), or the forward part of the invention.

When adding the feedback mechanism (which is just adding the fact that the human body must be stable, except when it is hit by cancers and a few more specific cases), then the production through metabolism of a Substance must be slowed down, when the Substance is abundantly available; so the invention assumes that the Genes involved in producing the Substance must be downregulated, since they are the only factors that control the production steps. In other words, at least one of the Genes involved in promoting the Metabolization Reactions of that Substance is downregulated. This can happen e.g. if you ingest that Substance. We refer to this as (C) or the feedback part of the invention.

When (A), (B), and (C) are joined together by means of the approximation, the invention can from data calculate causalities and do predictions of what happens, when an input is changed.

The approximation is defined such that we can include the many data sources (by importing them as a copy or by reference) and create data to fill them, where data is missing. When including data, it has many different ways of specification, and it is beneficial to utilize the special ways of each source in order to fit it into the overall approximation. E.g. to use the peculiarities of a diagram from KEGG on a Signaling Pathway when coupling its Ligands and Receptors as well as its transcription together with the rest of the data.

The invention facilitates the discovery of causalities that are not evident today in that they are not part of the same pathway in focus by researchers.

One special case is that some genes have more than one role, e.g. affect more than one element of the model, and the consequence of this discovery, taking into consideration that totality of this invention, is not implemented in prior research results. E.g. the gene DDC is involved in both three Metabolization Pathways and in a Signaling Pathway cf FIG. 26:
(A) The gene DDC is involved in three Metabolization Pathways including the process of producing Serotonin (and in subsequent steps Melatonin) from Tryptophan
(B) The enzyme corresponding to the gene DDC is a Coactivator to the Androgen Receptor (AR, which is a Transcription Factor) and thus must be present, if AR is to mediate its function—which is in part to cause Prostate Cancer. Therefore, if DDC is downregulated in a Prostate Cancer patient, the cancer will reduce its growth and spread.
(C) Due to the Feedback Mechanism, if e.g. Melatonin is ingested and thus is in ample supply in the body, the DDC gene may be downregulated. It is indeed mentioned in several references that ingesting Melatonin may reduce the probability and/or worsening of prostate cancer.

The example shows that the invention provides input to hitherto uninvestigated causalities that may lead to novel cures and treatments for diseases.

The uses and benefits of it include the areas of
Research into drugs, and causes and treatments for diseases such as cancer. Research will have an easier job of interpreting results, making tests etc., and research will be directed to further clarify what the model suggests
One special case of this is the classification of interactions between genes, which are amply recorded but not explained at present, into interactions that are “explained” by the approximation, and interactions that warrant further study, possibly the addition of data, to explain them.
Pharmacology i.e. the ability to describe the effects of drugs, thereby extending the functionality of clinical systems that advise on the optimum use of drugs

### Technical Field

The invention is implemented into a standard IT system with a database and associated functionality in an application.
The approximation is represented in a relational database with tables and relations
The functionality is implemented as one or more applications on top of this database, i.e. making use of its data to determine its functionality, which becomes data driven. One widespread use of applications is database queries
The functionality may be implemented in the same IT environment as the database, or remotely via integrations with the database or other applications based on it
When adding data, the said data enters the database as records obeying the data model
When adding functionality, additional tables may be added and populated with data, and additional applications may be added

### DESCRIPTION

### Brief Description of the Drawings

FIG. 1: The Human Body and its many functions as data with (data driven) applications on top of this data exemplified by the blood pressure system, “Renin-Angiotensin-Aldesterone System” or “RAAS”.

FIG. 2: Overview of the structure of the forward part of the invention. Numbers in brackets estimate the amount of each element in a human, these numbers not being a part of this invention: 20,000 genes, 2,200 metabolic reactions, 800 receptors. 7.660 signaling substances, 1,600 transcription factors, and 300 coregulators.

FIG. 3: The structure of the invention with table relationships indicated. Numbers explained: 1: Reaction acting on a hierarchy of substances (called an ontology). 2: Cascaded reaction. 3: Relationship of substances with Ligands that are in a hierarchy. 4: Relationship of ligands with receptors that are in a hierarchy called Families. 5: If the receptor is of the type “nuclear” it invokes expression as a transcription factor. 6: The relationship between receptors and signaling substances—both in hierarchies called families. 6a and 7: The relationship between signaling substances (and other signaling substances in 6a) and transcription factors (in 7)—both in hierarchies called families. 8: The relationship between transcription factors and expression. 9: Co-regulators can be involved with nuclear receptors and other transcription factors in expression—they are in a hierarchy called families. 10: Expression as a formula implicaling genes. 11, 12, 13, 14, and 15: Relationships (how they govern) between genes and elements involved in signaling, dashed for 12 and 15, if the element is not a Protein. 16: Feedback mechanism (for completeness, since it is not explained on the figure).

FIG. 4: Perception of the problem as a hierarchy of Genomics, Transcriptomics, Proteomics, and Metabolomics (Source: https://en.wikipedia.org/wiki/Genomics)

FIG. 5: The blood pressure system, called the Renin-Angiotensin-Aldosterone-System (or RAAS). The overall system, as a combination of metabolization and signaling —happening in different body parts.

FIG. 6: Overview of the two types of processes in the Human Body: (1) Metabolization with Reactions, and (2) Signaling starting from Receptors.

FIG. 7: Metabolization processes (Reactions). An except from a graphical overview of the 2,200 reactions.

FIG. 8: Signaling Pathways. From Receptors (at the boundary or membrane of a cell or accessible at the inside, because the ligand penetrates the membrane into the cell) through the Signaling Substances into the nucleus where Transcription Factors facilitate the gene expression

FIG. 9: Classification of Receptors into 5 types—in the context of two other classifications (where 1: “Ion Channel Receptors” are a subset of “Membrane Transports”, and where 4: “Nuclear Receptors” are a subset of “Transcription Factors”). The boxes without text inside are examples of non-receptors in these other classifications. The “Membrane Receptors” do not include 4: “Nuclear Receptors”, since they are located inside the cell, but ligands from outside the cell can reach them anyhow, since the ligands can penetrate the cell membrane.

FIG. 10: The overview in FIG. 2 together with FIG. 7 and FIG. 8 as well as with the addition of other parts of this invention comprising (1) Body parts, (2) Medication, (3) DNA Damage+Mutation, and (4) Immmune System.

FIG. 11: Metabolization (adding detail to a part of FIG. 10): Reactions and Substances

FIG. 12: Signaling (adding detail to a part of FIG. 10): Ligands, Receptors, Signaling Substances, and Transcription Factors. Functions

FIG. 13: Functions. Interim specification of signaling—and grouping, before it is diagrammed into signaling diagrams as data.

FIG. 14: Signaling (adding detail to a part of FIG. 10): Signaling Substances (incl. Receptors, Transcription Factors, and Coregulators) and their role in Expression of genes (Nuclear Receptors and (other) Transcription Factors, sometimes with Coregulators).

FIG. 15: The three tables that hold expression relations (from FIG. 14)—with coregulators (one shown)

FIG. 16: The three tables that record expression relations (from FIG. 14)—with coregulators (one shown)—reorganized for a better overview

FIG. 17: Signaling diagram example (Prostate Cancer from KEGG). Excerpt

FIG. 18: Signaling diagrams as data (adding detail to a part of FIG. 10). Arrows become relations in a table (the three tables that correspond to relations at the family level are shown)

FIG. 19: Signaling diagram example (excerpt). Arrows as data (on the family level) (from FIG. 18). Families broken down into their gene relations. Relationship of the diagram as a URL and the data.

FIG. 20: Gene based overviews. Signaling substances and pathway diagrams

FIG. 21: All tables of the invention. This diagram extends FIG. 10 and combines FIG. 11. FIG. 12, FIG. 14, and FIG. 18

FIG. 22: Hierarchy of Receptors

FIG. 23: The invention on a high level

FIG. 24: Key parts of the blood pressure system (RAAS) as represented in the invention

FIG. 25: Metabolization and Signaling together, showing the example where a substance in the Petabolix+zation Pathway—Serotonin—invokes Signaling by activating a Receptor, thus creating a branch in the overall approximation, where Serotonin can continue along two separate paths

FIG. 26: Feedback and Forward models together with the example of the DDC gene, with its role in Metabolization (and thus a role in the Feedback mechanism) as well as its role in a Signaling Pathway for prostate cancer

FIG. 27: Signaling Diagram and shortcut to getting the Receptors and thus its Ligands, as well as a shortcut to getting the transcription information using “DNA” on the diagram

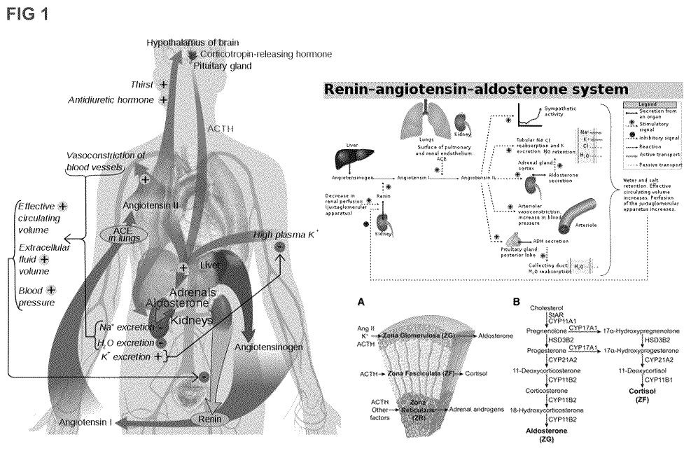

### DETAILED DESCRIPTION OF THE INVENTION

In this disclosure we present an invention that consists of a Data Model and Functionality using it, which approximate human functions in sofar as they are governed by the genes.

### The Forward Part

The forward part of this invention joins two areas in an end-to-end Pathway that combines the following areas in consecutive steps, brances, and joins of steps:
(A) Metabolization, defined as a (set of) reaction(s) that transfers one set of substances into another (different) set of substances, facilitated by one or more enzymes, releasing or consuming energy. Each enzyme tied to one gene.
(B) Signaling, defined as the cascaded set of actions without translation of substances, where one element is triggered by another element, initiated by receptors acting on ligands activating them, through to expression of genes. These elements are themselves governed in part by genes, so that expression is a function back to other genes.

Genes determine enzymes (in a 1:1 relationship). Genes affect Metabolization and Signaling in the following ways:
They generate enzymes (through Diplotypes) that catalyze all Metabolization processes
They control (through Diplotypes) the appearance and behavior of all Receptors, some Signaling Substances (those which are proteins), all Transcription Factors, and all Coregulators

The Diplotypes may differ in effect and effectiveness depending on which Alleles they are made up of, and thereby which mutations have occurred in the Genes forming these Alleles.

The metabolization reactions produce ligands that affect signaling through receptors, which in turn regulate the transcription of other genes. In some cases the ligands are proteins directly produced by genes, i.e. the ligands don't have to wait for a metabolization to occur.

As an example, the blood pressure system (the “Renin-Angiotensin-Aldosterone System” or “RAAS”) is a mix of the two areas, one after the other, concatenated: As a subset cf FIG. 24
1. The “REN” enzyme/gene (Renin) catalyzes a metabilzation reaction converting “Angiotensinogen” to “Angiotensin I”.
2. The “ACE” enzyme/gene catalyzes a metabilzation reaction converting “Angiotensin I” to “Angiotensin II”.
3. “Angiotensin II” then triggers (is a ligand to) several receptors e.g. the “Type-1 angiotensin II receptor” (governed by the “AGTR1” gene) affecting several cascaded signaling steps that end up in the increased expression of the gene “CYP11B2”.
4. In a cascaded series of metabilzation reactions “Cholesterol” is converted to “Aldosterone” catalyzed by the enzyme/gene “CYP11B2” (as a chief enzyme).
5. “Aldosterone” triggers (is a ligand to) the “Mineralocorticoid receptor” (a nuclear receptor) which through increased transcription of certain genes regulates the amount of Sodium (Na) and Potassium (K), and thus the blood pressure.

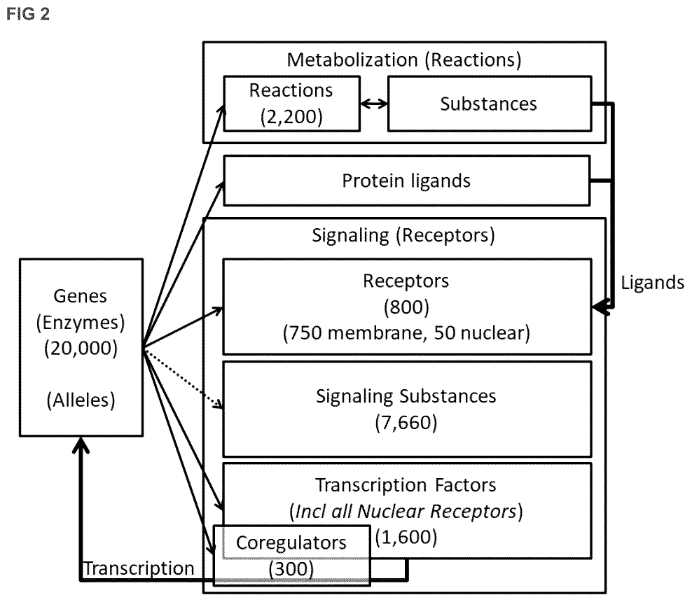

According to the invention tables of a database are produced that hold and represent (cf. FIG. 2)
Genes and their relationship to definitions like UniProt
Reactions and their relationship to the EC numbering etc.
Substances—and thir relation to PubChem as well as to medicine, if they act as active substances as well
The relationship of Reactions with Genes and with Substances as input and output respectively
Ligands
How the Substances or Genes relate to the Ligands
Receptors and their relationship with Genes and with Ligands
Signaling Substances
Transcription Factors
Coregulators
How the Receptors, Signaling Substances, Transcription Factors, and Coregulators relate together and how they are governed by the Genes to facilitate a Signaling Pathway and perform the transcription—and of which other Genes, probably not those that govern the elements

### Hierarchies

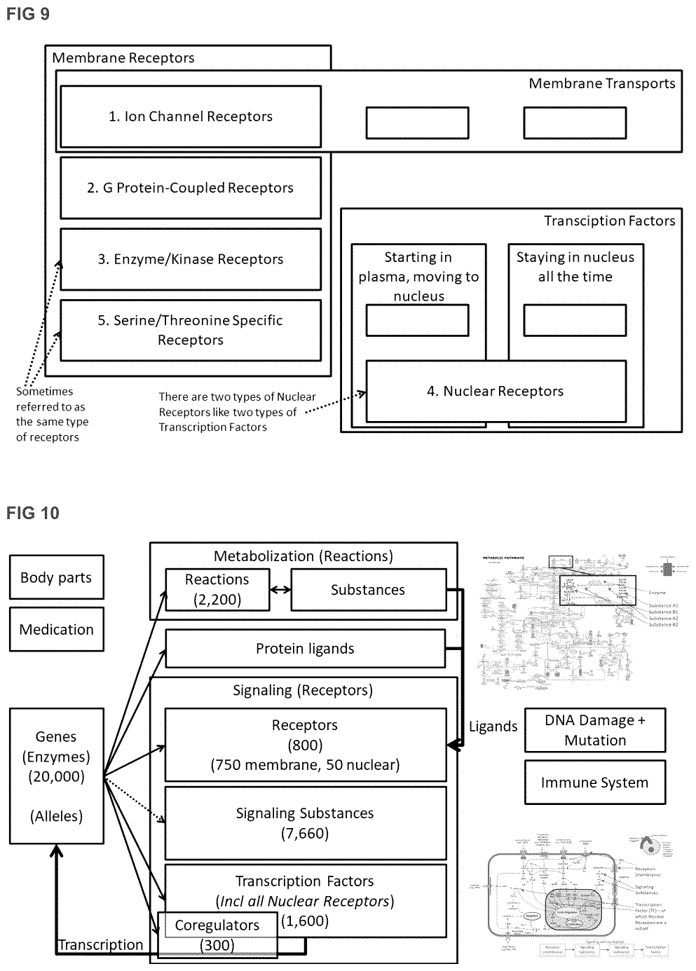

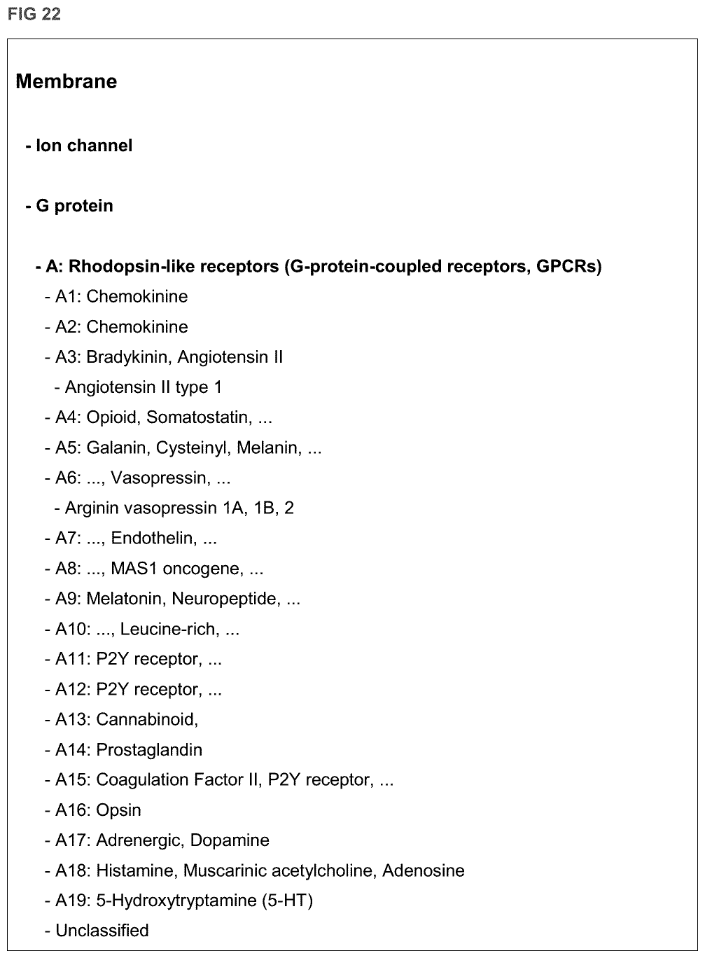

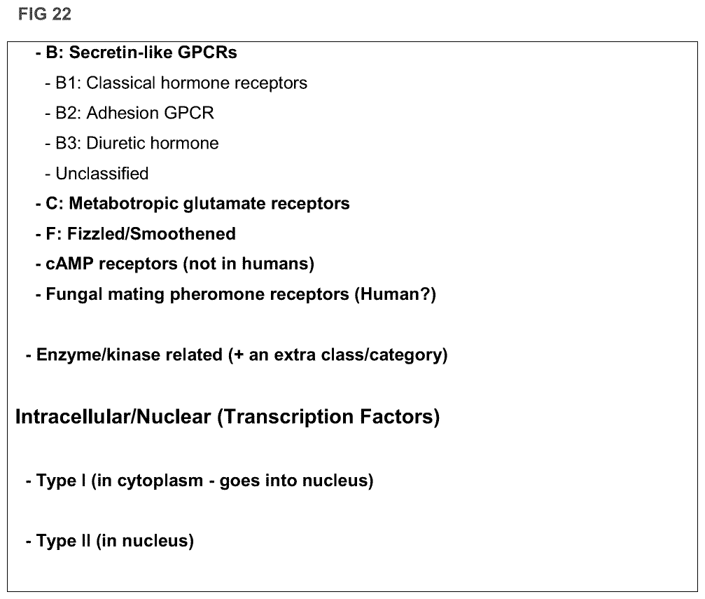

Some functionality acts on different hierarchical levels (except proteins and their 1:1 correspondence with genes). This implies that the approximation holds hierarchies of:
Substances—since a reaction may act on substances up in a hierarchy (i.e. on all the substances that are positioned below in the hierarchy)
Ligands—which may be defined as a number of substances, where the substances can be on a higher hierarchical level or the lowest level having a PubChem ID
Receptors having multiple levels from the top level down to at least 800 at the bottom level, where each receptor is a protein governed by a gene. See FIG. 9 for the top level of the hierarchy as well as its overlap with transcription factors and other classifications and FIG. 22
Signaling Substances having multiple levels
Transcription factors in multiple hierarchies having multiple levels each
Coregulators having multiple levels

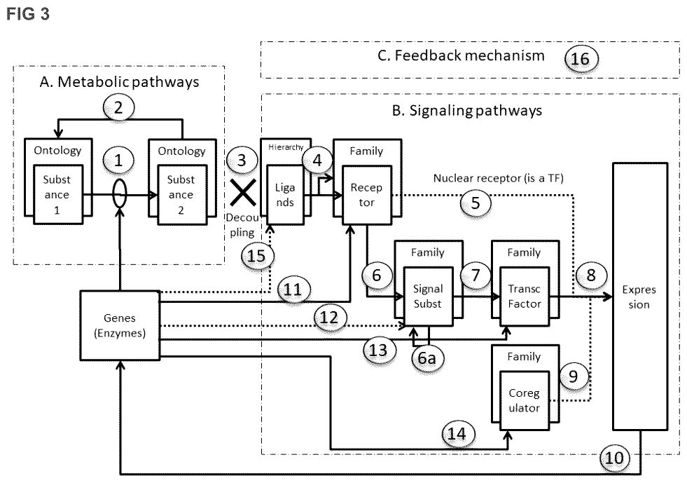

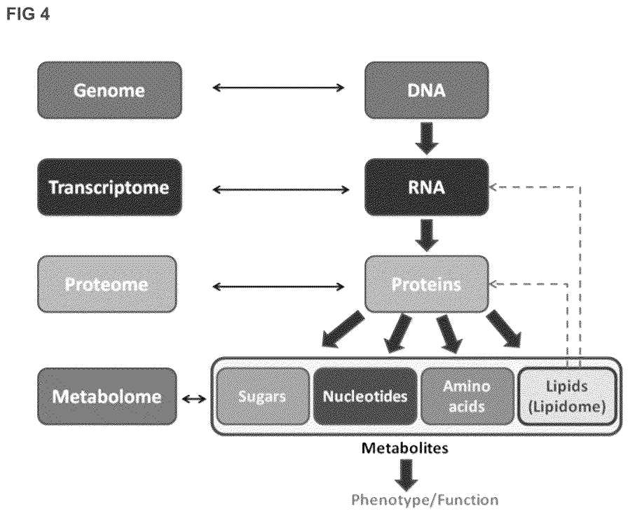

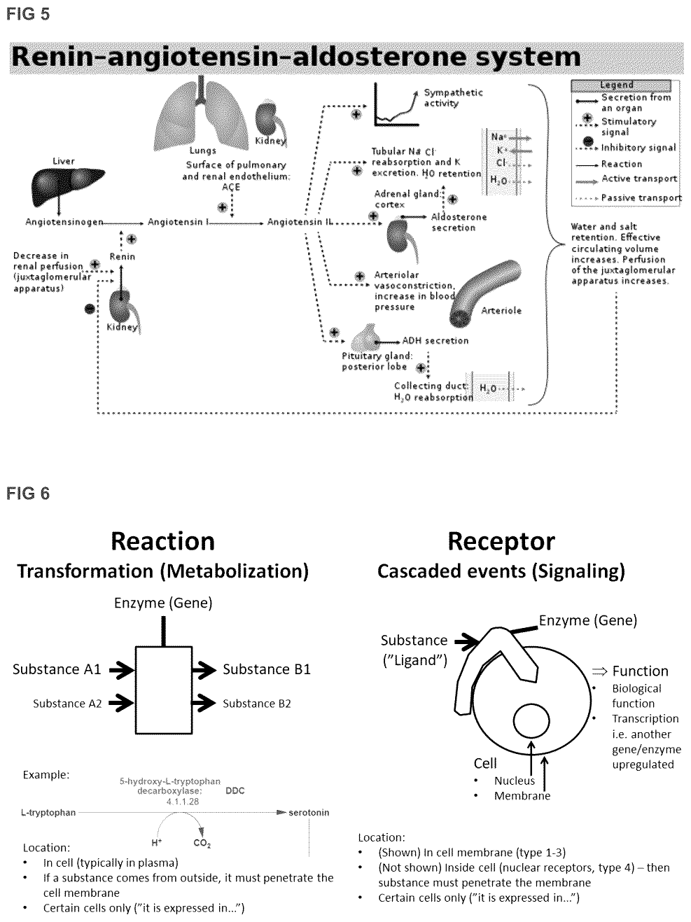

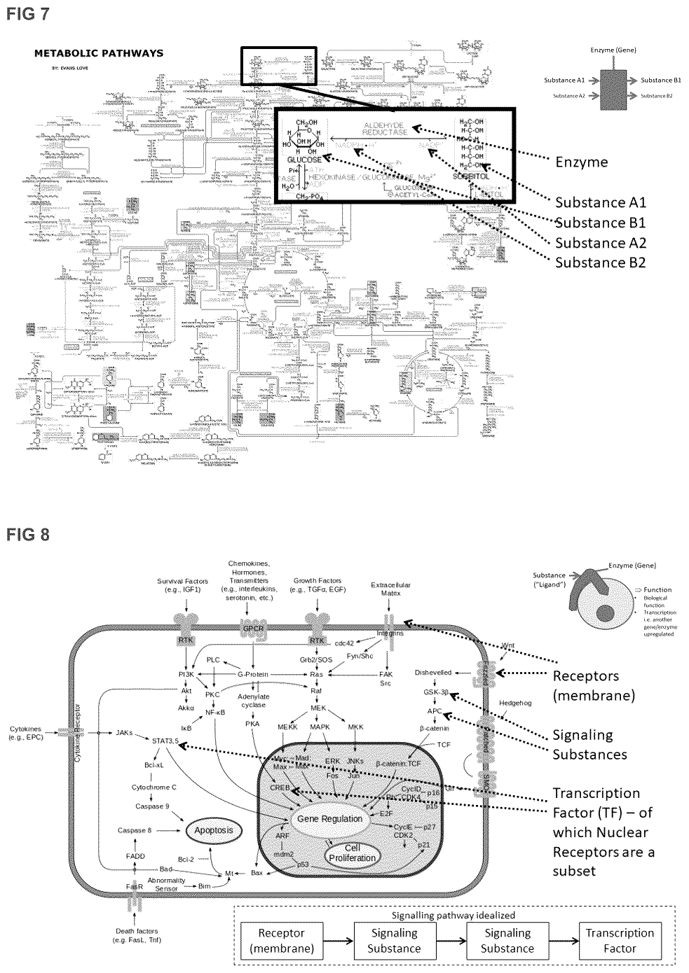

The drawing in FIG. 3 explains the tables in the context of the hierarchies and with mention of the Feedback mechanism as well. Numbers on the figure are explained in the description of the figure.

An element in the approximation must relate to another element cf FIG. 3, and the two elements can be in any of the hierarchical levels. This occurs in the following situations:

### The Relations Between Elements on Different Levels in a Hierarchy

Reactions to Substances (Substances can be higher-ups in the ontology hierarchy)
Substances to Ligands
Genes to Ligands (where the genes are not part of a hierarchy)
Ligand effects on Receptors (Ligands can be from different hierarchical levels, and the Receptors can themselves be from different hierarchical levels)
Receptors to Signaling Substances
Receptors to Transcription Factors
Signaling Substances to Transcription Factors
Transcription Factors and Nuclear Receptors and Coregulators to Transcription Rules (Transcription Factors can be from different hierarchical levels, but the Genes are never grouped in a hierarchy)

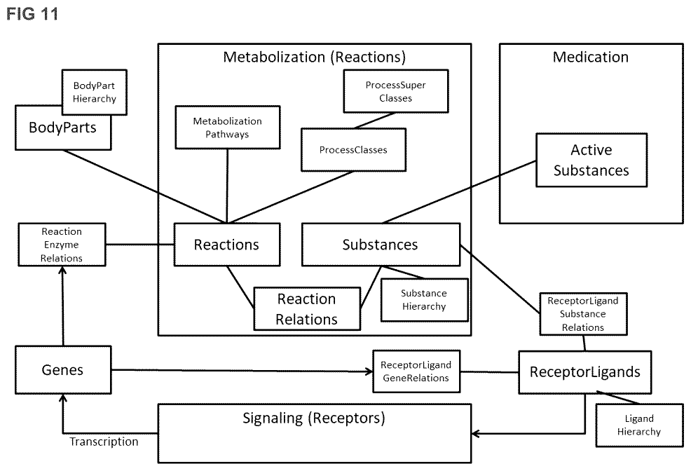

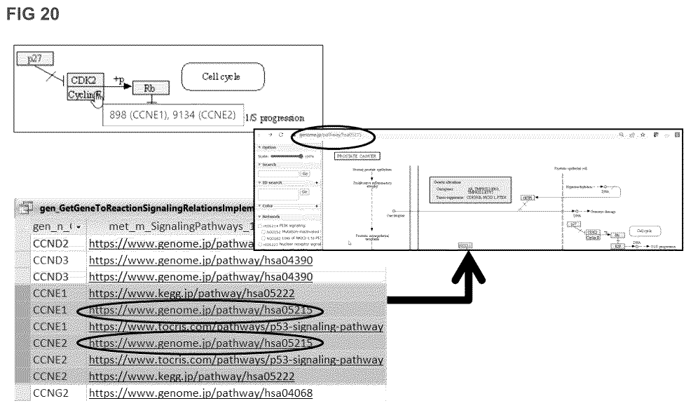

Drawings that describe this set of tables and their relations, and the definition of Functions and their association to Receptors (which latter part is not show in FIG. 3), are included in FIG. 11 up to FIG. 20.

### Relation Types

Ligands have effects on a Receptor ranging either as a continuum or enumerated as the following list, implemented in the table that relates the Ligands with Receptors cf FIG. 3, point 4:
Super Agonist
Full agonist
Partial agonist
Silent antagonist
Partial antagonist
Full antagonist
Positive allosteric modulator
Negative allosteric modulator

Signaling cascades, implemented in the tables that hold the relationships cf FIG. 3, points 4 up to point 10, have at least the following types—with more to be included—which are currently implemented as arrow types in the existing signaling diagrams:
Activate, Stimulate, or Upregulate in a single step or a multi step
Inhibit
(Activate or Inhibit) may be combined with Methylate, Phosphorylate, Ubiquinate, Glycolysate
(Activate or Inhibit) may be combined with De-methylate, De-phosphorylate, De-ubiquinate, De-glycolysate
Expression, Repression
Missing [interaction] by mutation
Binding/association, Dissociation
Indirect, Unknown
Translocate

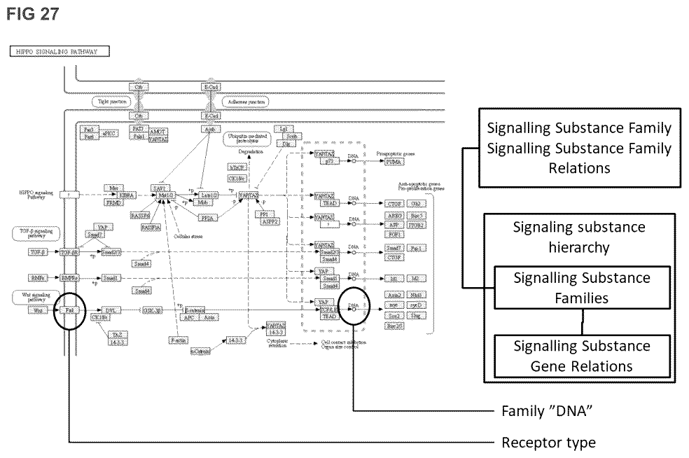

Gene relations: the effect of a mutation cf FIG. 3, points 11 up to point 15, is in some diagrams implemented as a relation type (an arrow in the diagram) cf FIG. 3, point 10—see the point on the Family “DNA” in FIG. 27. They are generally implemented through the effect of pairs (Diplotypes) of Alleles (instances of genes)—and the transfer function including the effect of different mutations (different Alleles) is defined to be any function

Some genes have more than one role, e.g. affect more than one part of the approximation cf FIG. 3, points 11 up to point 15.

### Statistics of Mutations and DNA Repair

The statistical models incorporated in this invention to cover the effect of mutations of Genes and their effectsv cf FIG. 3, points 11 up to point 15 and cf. FIG. 10 include
Mutations already instantiantiated and inheritable
Error occurrence functions incl. frequence and likelihoods in cell division
DNA repair functions—and their relation to genes and already occurred mutations like in the BRCA2 gene governing DNA repair and implicated in breast cancer—incl. their reactions to being over-burdened etc.
Mutations that pass the DNA repair functions
Likelihood (functions and their thresholds) etc. of mutations being discovered by the immune system or by the apoptosis functionality

### Speed, Branch, and Joins in the Pathways

This approximation explains cancers, taking into consideration that genes mutate, and some mutations survive the “DNA repair” functions, and then act as described in the approximation.

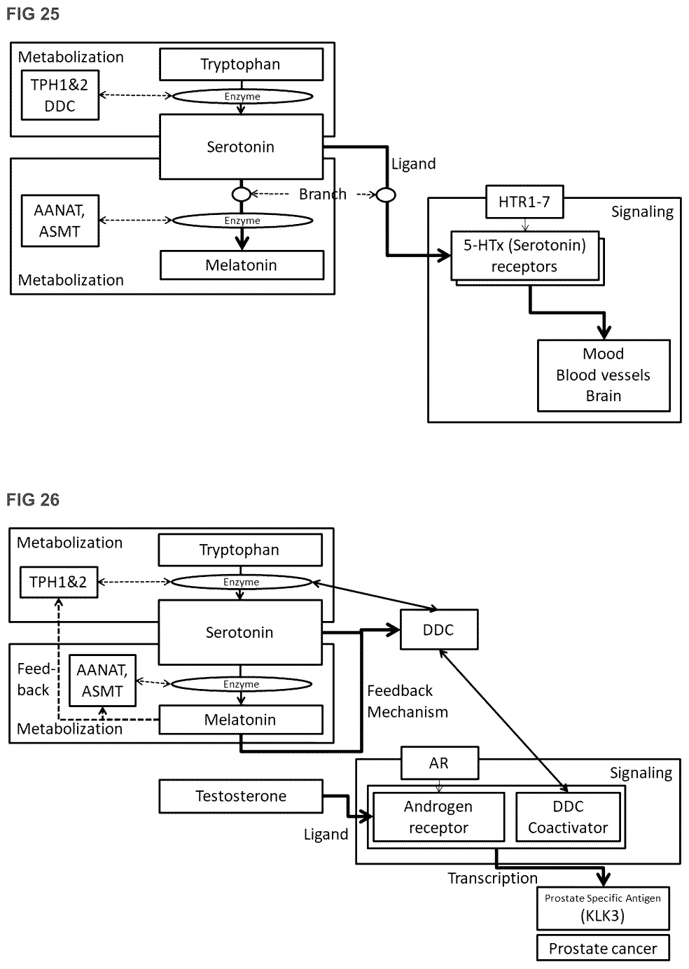

The invention covers the following aspects:
Speed and timing factors—and formulae with timing factors: There is no implementation currently of how fast a process happens
Distributions in pathway branches:
There is no implementation currently of by which percentage a pathway goes in one of several possible directions, when it branches out cf FIG. 25
There may furthermore be several unsaid conventions regarding a merge in diagrams: Is the outcome of each branch required for it to ge forward (an AND function) or is just one of the merging branches required (an OR function)—or a combination of that. We currently assume an AND function: That all inputs are required.

### Body Parts

The invention assumes that the functionality of metabolization and signaling is the same wherever it occurs, so that the differentiation between body parts and their different functions is covered by the initial distribution of certain enzymes and proteins that facilitate this signaling.

The initial such distribution is part of this invention, and it has a table and a hierarchy for Body Parts.

Until we get to a final result of this distribution and a complete set of data for the approximation, the invention holds a relationship between key elements and the body parts cf FIG. 10.

### The Immune System

The invention assumes that the functionality of the immune system is covered by the metabolization and signaling functions specified.

Until we get to a complete set of data for the approximation, the invention holds a relationship between key elements and the parts of the immune system cf FIG. 10.

### Aging

The invention assumes that aging is primarily driven by mutations and DNA repair functions—which are covered by the invention cf FIG. 10.

### The Feedback Part (C)

The invention covers a reverse metabolization relationship, where all the genes involved in the metabolization pathways that lead to the production (through conversions) of a substance are downregulated by that substance.

The invention does not point out which genes (if not all) and the details of that downregulation, just that it happens to one or several of the genes.

When properly described that feedback mechanism will probably become a forward (signaling) mechanism—but since this is today poorly described and not the priority of research, and since it may implicate functions and elements not part of the forward part of this invention, we simply refer to the “feedback mechanism”.

An example of joining the two mechanisms is mentioned above for the downregulation of the DDC gene by substances like Melatonin—according to the forward part of this invention leading to less prostate cancer in some situations.

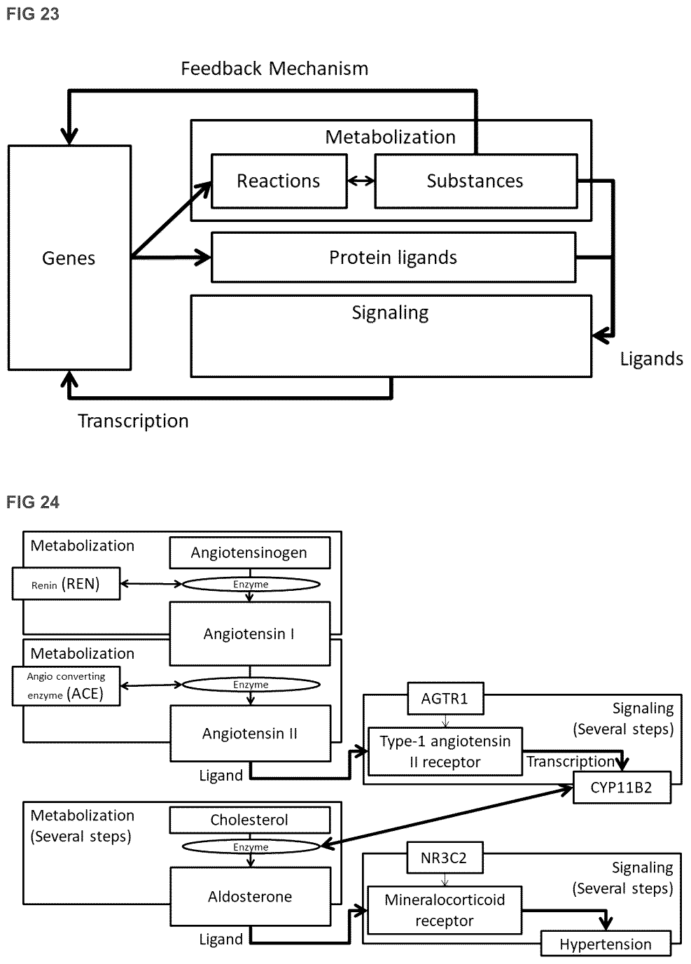

An overview of the complete invention is shown in FIG. 23. The example where DDC is downregulated by Serotonin and by Melatonin and then has a beneficial effect on prostate cancer is shown in FIG. 26.

### Functionality

When putting data together in approximation data model (forward and feedback, metabolization and signaling, statistics and thresholds) you get the opportunity to create functionality in these areas:

Overviews
When listing all approx. 20,000 genes, you can show what role(s) they each play (which elements they govern) and thereby discover the occurrence of several roles
Relationships by means of links etc. to external data and diagrams

Causality
When you add or increase a substance or express a gene more, or apply a mutation then you get more/less of a substance, gene, function, and/or a particular consequence will happen, across the whole human body. You can convert signaling diagrams to queries that explain this e.g. for the the blood pressure regulating system “RAAS”. This is used to asses body functions as well as the effect of drugs.
When taking into account time and speed functions as well as distribution functions at branch points and join functions, these causalities will become estimations of amounts and with timelines—and we can predict the fact that a branch “comes first” to its end, and assess its impact on other parts of the model.
We can classify the interactions between genes into those that are explained and those that are not—listing interactions that should be investigated further in order to shed light on functions of the human body.

Cancer Specifics
We can compute when instability occurs, i.e. when e.g. DNA repair and apoptosis is overwhelmed, and thereby when cancer happens, and thereby explain why it is happening. And we can use the model to see whether stability can be reinstated, thereby suggesting a way in which to treat the cancer.

Outstanding Lists
It is possible to derive e.g. the following identification of missing data:
Missing Receptors (e.g. from the total set of Receptor interactions)
Receptors without a Ligand
Unspecified Ligands (by gene or substance)
Missing signaling diagrams per Receptor (with or without functions)
Unspecified genes
Genes whose transcription is not defined

Since we have approximate numbers estimating the total amount of each element cf FIG. 2, we can estimate how far we are from having data for all Signaling (assuming that we have all Metabolization).

### Target for Data Aquisition

Target or boundary conditions for completeness: There are certain indicators of when we are done with the data aquisition:
All approx. 20,000 genes have at least one role (they regulate at least one element—or it is explained what else it does, if it does not affect the proteins of this model)
All known signaling elements (receptors, signaling substances, transcription factors, and coregulators) are included in the model
All functions that we know of in the human body, and which are metabolization or signaling dependent, are converted to at least one diagram
All genes are associated with at least one expression function

When the invention is fully implemented with regard to its data—it will be natural to extend the data model and thus continue and refine or update the mapping process. This is outside the scope of the present invention.

### BEST MODE FOR CARRYING OUT THE INVENTION

The invention can be implemented as the combination of
a relational (SQL) database, and
functionality associated with it in the form of
   SQL queries,
   rule implementations associated with the database, and
   other applications that are data driven, and whose functionality is governed by a database.

### Data and Data Model in the Database

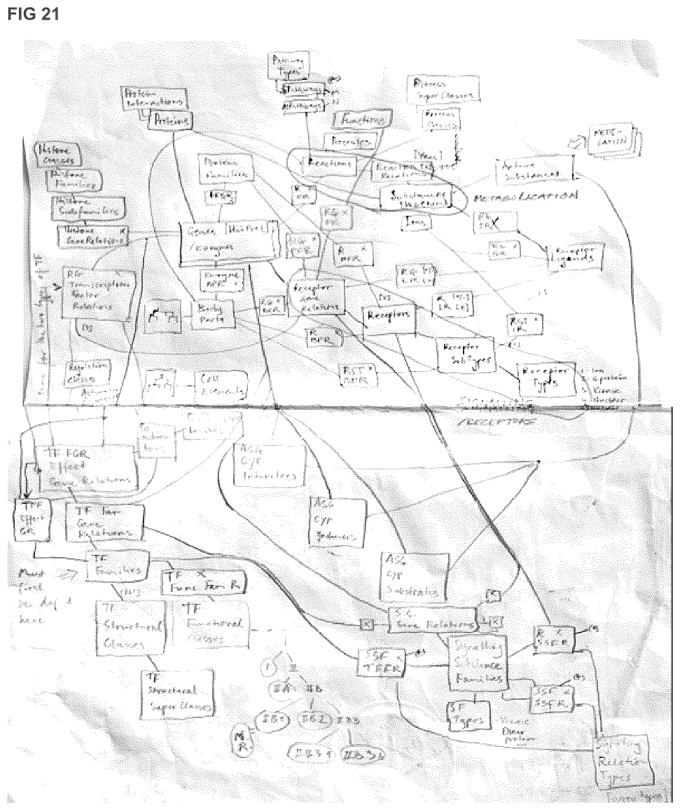

The full data model one implementation of the invention can be seen in FIG. 21.

It is a convention in the following that “Enumerated” means that the number in itself is significant, e.g. there are known to be a certain number (5) of receptor types, and we distinguish based on that number. When not “enumerated”, the content is just numbered for internal reference, but the number itself bears no significance.

The Forward Metabolization mechanisms—and implicitly also the Backward mechanisms—are recorded in tables where the table names are listed in the following: (See FIG. 11 for an overview of tables)

### Genes

Table names:
Genes (20,000—tied to UniProt with an ID)
ReactionEnzymeRelations then define which Enzymes/Genes relate to which Reactions

### Body Parts

Table names:
BodyParts (where all body parts relating to the model are recorded, no matter where in the hierarchy they are)
BodyPartHierarchy define which Body Parts are subsets of which other Body Parts—thereby defining the hierarchy

### Metabolization

Table names:
Reactions
Substances (tied to PubChem via an ID—unless they are higher up in the hierarchy [which is not shown])
ReactionRelations describing which Substances are in which Reactions, and whether they are Inputs or Outputs in the Reaction.
MetabolizationPathways (which group Reactions together)
ProcessClasses
ProcessSuperClasses [enumerated]

### Medication

Table names:
ActiveSubstances: If a Substance is also in the medication model as an active substance, we put a 1:1 relationship

### Ligands

Table names:
ReceptorLigands (listing all the Ligands used by one or more Receptors)
ReceptorLigand Substance Relations that for each Ligand defines it by the one or several Substances that are said Ligand. This is the main table that links Metabolization (having Substances as outcome) to Signaling (having Ligands as triggers)

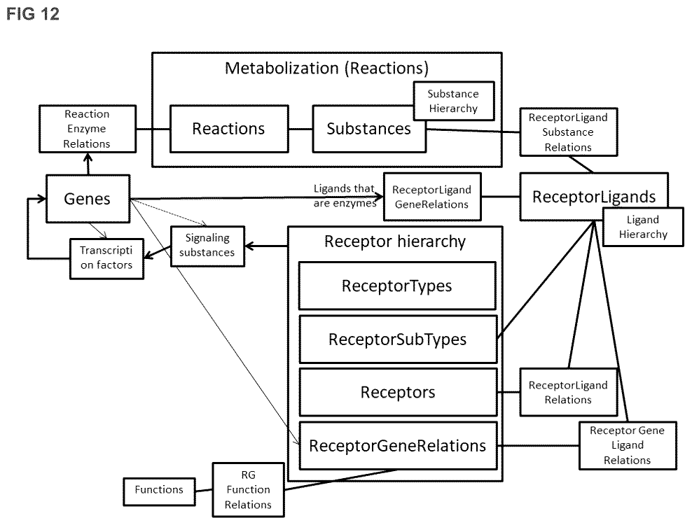

The Forward Signaling mechanisms and their relationship to Metabolization are recorded in tables as follows: (See FIG. 12 for an overview of tables)

### Receptors

Table names:
ReceptorTypes [enumerated]

### ReceptorSubTypes [see

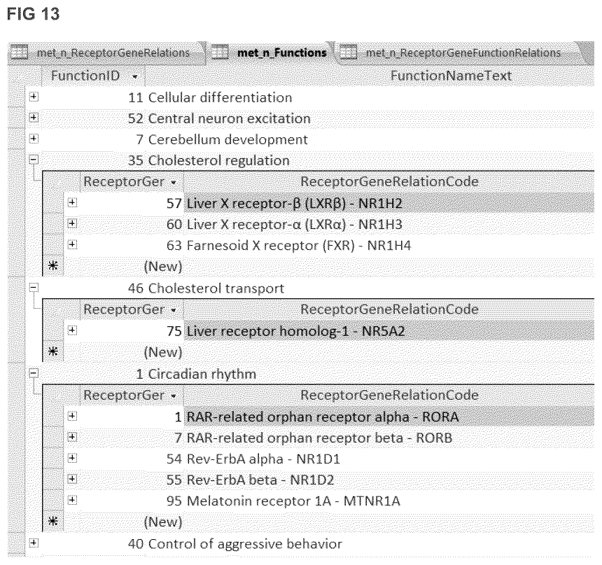

FIG. 22 for a sample structure of these entries in the table]
Receptors
ReceptorGeneRelations
ReceptorLigandRelations
ReceptorGeneLigandRelations
Functions (Specifying free text functions in the Human Body that explain what this Function consists of, e.g. “Circadian Rhythm” (the heartbeat).
ReceptorGeneFunctionRelations [see examples in FIG. 13]. These Functions represent effects that aren't described by strict data modelling—and they are foreseen to be replaced by strict signaling data in the future

### Body Parts

Table names:
ReceptorSubTypeBodyPartRelations
ReceptorBodyPartRelations
ReceptorGeneBodyPartRelations

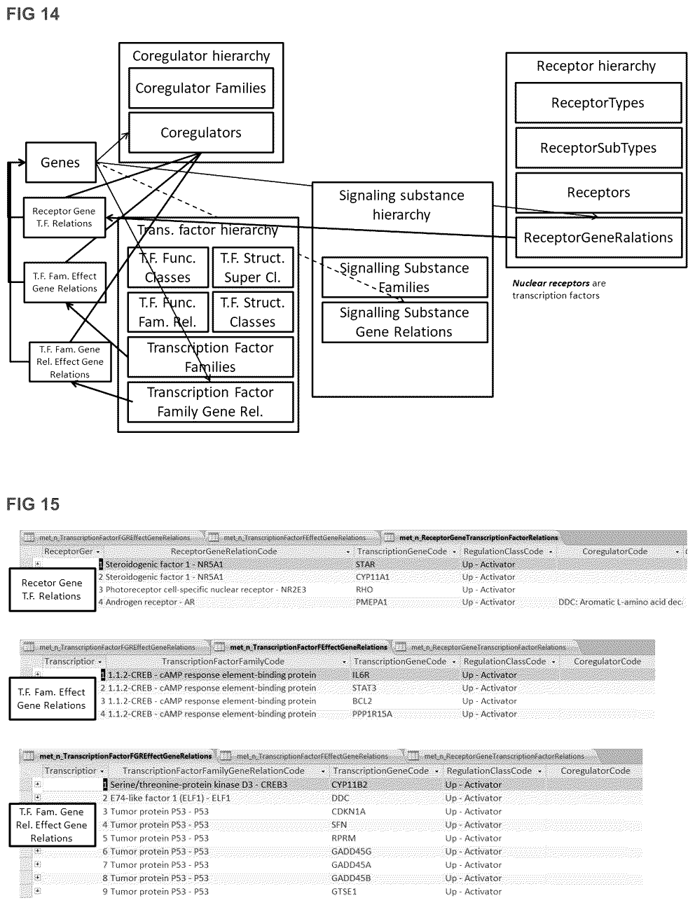

The Forward Signaling mechanisms and their relationship to Gene Expression are recorded in tables as follows: (See FIG. 14 for an overview of tables)

### Signaling Substances

Table names:
SignalingSubstanceFamilies
SignalingSubstanceGeneRelations

### Transcription Factors

Table names:
TranscriptionFactorFunctionalClasses [enumerated]
Transcription FactorFunctionalFamilyRelations
TranscriptionFactorStructuralSuperClasses [enumerated]
TranscriptionFactorStructuralClasses
TranscriptionFactorFamilies
TranscriptionFactorFamilyGeneRelations

### Genes (Recording the Expressions—See Examples in FIG. 15:)

Table names:
ReceptorGene TranscriptionFactorRelations
TranscriptionFactorFamilyEffectGeneRelations
TranscriptionFactorGeneRelationFamilyEffectGeneRelations

It is part of the invention that if a Gene is mentioned multiple times, there is by default an OR rule between them—and if this is different, then there is an entry in [the table that handles multiple Expressions rules for one Gene]

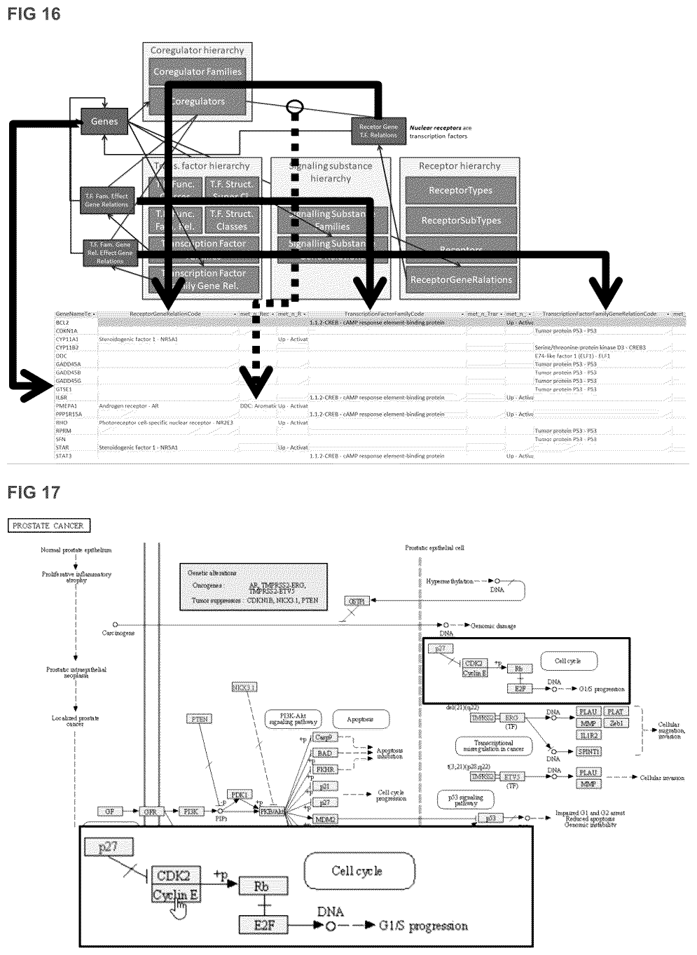

The overview (where only one Expression per Gene is shown) is further depicted in FIG. 16.

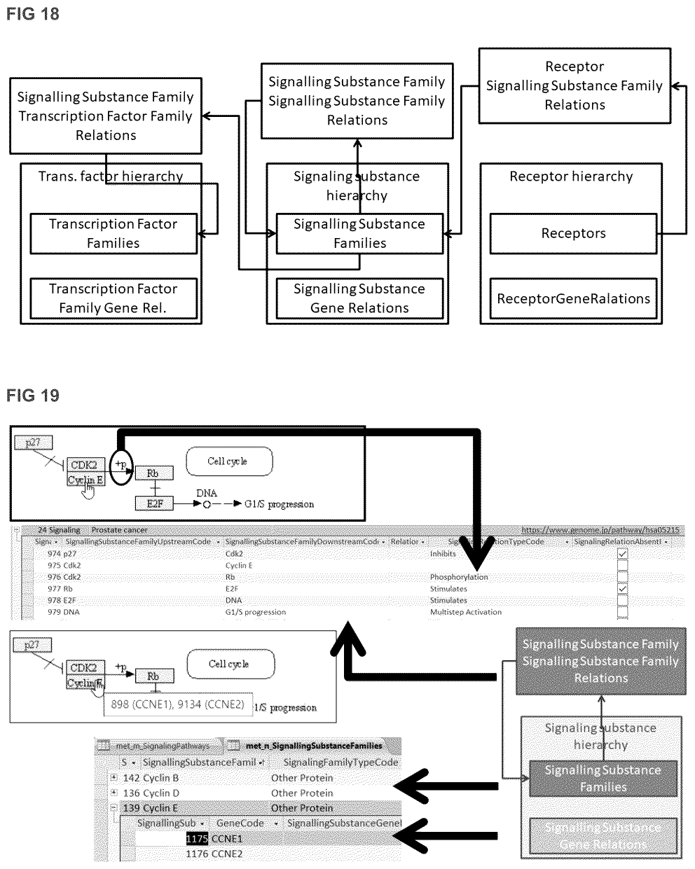

The Forward Signaling mechanisms and their relationship to Signaling Diagrams are recorded in tables as follows: (See FIG. 17 for an example of a Signaling Diagram and FIG. 18 for the overview of the data model)

### Receptors to Signaling Substances

Table names:
ReceptorSignallingSubstanceFamilyRelations
[And one table for each combination of hierarchical level]

### Signaling Substances to Signaling Substances

Table names:
SignallingSubstanceFamilySignallingSubstanceFamily Relations.
   This is where the diagrams are entered in the beginning—also if the one or both of the Signaling Substances involved is a Receptor or a Transcription Factor. They can later be bound to the right Receptor or Transcription Factor by moving the record to the right table.
   We use the upper level in the hierarchy (Families) to have the freedom to associate the Element to one or several Genes. See FIG. 19.
[And one table for each combination of hierarchical level]

We can then use that set of relations to provide an overview of which Signaling Pathways a Gene is related to, and link to that pathway (see FIG. 20).

### Signaling Substances to Transcription Factors

Table names:
SignallingSubstanceFamilyTranscriptionFactorFamilyRelations
[And one table for each combination of hierarchical level]

In many diagrams (e.g. from KEGG) the Expressions relationship is given through a “DNA” Element in the diagram. We have entered “DNA” as if it were a Signaling Substance Family—when making a more detailed recording this record can be moved to the appropriate table and the corresponding right formula can be entered e.g. in the table TranscriptionFactorFamilyEffectGeneRelations. See FIG. 27.

### Immune System

The Immune System is [at present] handled through its Signaling Pathways.

The current implementation of the invention does not yet include the following aspects, but the invention covers the following aspects:
Speed and timing factors—and formulae with timing factors:
There is no implementation currently of how fast a process happens
Distributions in pathway branches:
There is no implementation currently of by which percentage a pathway goes in one of several possible directions, when it branches out
There may furthermore be several unsaid conventions regarding a merge in diagrams: Is the outcome of each branch required for it to ge forward (an AND function) or is just one of the merging branches required (an OR function)—or a combination of that. We currently assume an AND function: That all inputs are required.

### Mechanisms—Forward/Feedback

The Forward mechanism is described above: Triggered by Genes (as Enzymes) Reactions produce Substances that as Ligands activate Receptors that activate Signaling Pathways that besides fulfilling Functions end up in in- or de-creasing Gene Expression (transcription).

The Feedback mechanism does not require a separate recording:
Substances feed negatively back on the Genes/Enzymes that produce them
These mechanism may later be explicitly recorded e.g. as Signaling
   Functionality (as SQL Queries and/or Data Driven Applications Associated with the Database)

A lot of queries can be made by means of SQL queries on the data model given by the approximations.

1. A Method for establishing an approximation of the processes in a human body, implemented in a database, said Method comprising from one to an unbound number of the steps of the following Types ((A), (B), and (C)):
(A) Metabolization step Type, where Substances are converted to other Substances as related to Genes,
(B) Signaling step Type, where said Substances or Proteins related to Genes, make an association with a Receptor related to Genes leading to the activation of said Receptor, which through a cascade of steps and events facilitates Functions in the body as well as transcription and expression of genes,
where the said step Types (A) and (B) are combined into a Pathway, and
(C) Feedback step Type, where the combinations of (A) are reversed to point out which Genes are involved in the production of a Substance and downregulated by the said Substance, when the amount of said Substance increases,
such that it is possible to compute Causalities between Genes and Substances and Functions in the human body.

2. A method according to claim 1, wherein the Metabolization step Type (A) consists of a Reaction, where one set of Substances is converted to another set of Substances, where each of the said Substances is either a single chemical substance defined by e.g. an identifier like the identification code in PubChem, or the said Substances are elements of a substance hierarchy, said hierarchy being of the type many-to-many, where the bottom level of said hierarchy consists of chemical substances, said Reaction promoted by one or several Enzymes, each such Enzyme governed by a Gene through its pairs of instances, said instances called Alleles, said pair called a Diplotype.

3. A method according to claim 1, wherein the Signaling step Type (B) consists of:
a Ligand, said Ligand defined by zero, one, or several of said Substances in combination with zero, one, or several of said Enzymes, with at least one Substance or one Enzyme,
said Ligand being defined as elements of a ligand hierarchy, said hierarchy being of the type one-to-many,
said Ligand relating to a Receptor, the relation being called an Activation of the Receptor, said Receptor having from one to an unbound number of Ligands, said Activation classified either as a continuum or enumerated reflecting the role and the strength of the Activation,
said Receptor being elements of a receptor hierarchy, said hierarchy being of the type one-to-many, where the bottom level of said hierarchy is a Protein relating to a Diplotype and governed by a Gene,
said Receptor invoking either
a Function, which describes in words what the Effect of the Signaling is, or
a set of Relations called a Signaling Pathway between Elements of the following types, or both,
from zero, zero required if the Receptor is of the type Nuclear Receptor, to an unbound number of Signaling Substances, defined as a Substance or a Protein (said Protein relating to a Diplotype and governed by a Gene) or an Event external to the human body e.g. stress, radiation, or heat shock,
from zero, zero if the Receptor is of the type Nuclear Receptor, otherwise from one to an unbound number of Transcription Factors, defined as a Protein (relating to a Diplotype and governed by a Gene), which mediate the Transcription on one or more Genes, without or in a relation with the following
from zero to an unbound number of Coregulators (relating to a Diplotype and governed by a Gene), which mediate the said Transcription of one or more Genes together with one or more Transcription Factors, according to a Boolean function: either positively, in which case the said Coregulator is called a Coactivator, or negatively, in which case the said Coregulator is called a Corepressor,
and where the said Relations between said Elements of the Signaling Pathway describes the nature of the said Relation,
and where the said Transcription lead is to the upregulation or downregulation of the said Genes.

4. A method according to claim 3, wherein the said Activation of a Receptor by a Ligand if classified by an enumeration has a classification as one of the following
Super Agonist
Full agonist
Partial agonist
Silent antagonist
Partial antagonist
Full antagonist
Positive allosteric modulator
Negative allosteric modulator.

5. A method according to claim 3, wherein the Relations between said Elements of the Signaling Pathway is one or several of the below relation types:
Activate, Stimulate, or Upregulate in a single step or a multi step
Inhibit
(Activate or Inhibit) may be combined with Methylate, Phosphorylate, Ubiquinate, Glycolysate
(Activate or Inhibit) may be combined with De-methylate, De-phosphorylate, De-ubiquinate, De-glycolysate
Expression, Repression
Missing [interaction] by mutation
Binding/association, Dissociation
Indirect, Unknown
Translocate.

6. A method according to claim 1, wherein the step Types (A) and (B) are combined into a Pathway in one of the following ways
concatenations (one step type after the other),
with branches (two or more step types in parallel, each branch continued separately), and
with joins (two or more step types that are followed by one step type).

7. A method according to claim 1, wherein some of the Substances are exogenous, i.e not naturally occurring (e.g. drugs and poison).

8. A method according to claim 1, wherein the addition of a Substance, already in the human body or exogenous, causes an effect calculated with the use of the Causalities.

9. A method according to claim 1, wherein Functionality, that take all the variables mentioned as input parameters, is used in the following extensions to the method:
Timing Functionality in each Metabolization step and each Signsaling Relation,
Distribution Functionality among branches (with a special case being distributions adding up to 100%),
Join Functionality taking into account Timings and joining logic having Boolean functions as special case.

10. A method according to claim 1, wherein the functionality of relating to “a Diplotype and governed by a Gene” involves calculating the statistics of Gene mutations given inheritance of known mutations incl. mutations associated with an inherited disease and cross-likelihoods between two diseases, hereunder
the statistical distribution, given mutations already inherited and other conditions,
the passing of thresholds applied in DNA repair functionality,
applied through Alleles and their pairing in Diplotypes.

11. A method according to claim 10, wherein the DNA repair functionality doesn't catch and reverse a Mutation, which therefore persists, and the effect of it on the human body is assessed in terms of its effects on the relationship between the Genes and their corresponding Enzymes in Metabolization and their corresponding Proteins in Signaling Pathways.

12. A method according to claim 11, wherein Thresholds for instability are calculated or estimated, related to the Mutations (e.g. the proliferation of cells gets out of control due to Thresholds for apoptosis or other immune system mediated cell death being passed) thereby causing diseases like cancer.
